(19) United States
(12) Patent Application Publication
(10) Pub. No.: US 2024/0194333 A1
(43) Pub. Date: Jun. 13, 2024

(54) Replacing data points in a sequence of peak-to-peak interval calculations

(71) Applicant: Plume Design, Inc., Palo Alto, CA (US)

(72) Inventors: Ta-Lung Lee, New Taipei City (TW); Zhicheng Qiu, Cupertino, CA (US)

(21) Appl. No.: 18/079,090

(22) Filed: Dec. 12, 2022

(51) Int. Cl. G16H 40/63; A61B 5/00; A61B 5/024

(52) U.S. Cl. CPC G16H 40/63; A61B 5/02427; A61B 5/7278

(57) ABSTRACT

Processes for correcting time interval calculations in a dataset are provided. A method, according to one implementation, includes obtaining a sequence of data points (x) representing time interval calculations (y), each time interval calculation (y) based on a length of time between two successive peaks (p) in a photoplethysmography (PPG) waveform, each peak (p) presumed to represent a volumetric blood-flow peak during one of multiple cycles of the PPG waveform. In response to detecting one or more spurious data points in a segment of the sequence, the method includes creating one or more artificial data points for replacing the one or more spurious data points, wherein the one or more artificial data points are derived using the time interval calculation (y) of each of the one or more spurious data points. In some cases, the method may be performed by a smart ring having a PPG sensor and processing device.

### FIELD OF THE DISCLOSURE

The present disclosure generally relates to measuring heartbeat using Photoplethysmography (PPG) technology. More particularly, the present disclosure relates to systems and methods for detecting and correcting spurious data points related to PPG peak-to-peak intervals caused by noise and movement artifacts associated with a PPG sensor, such as a PPG sensor incorporated in a smart ring.

### BACKGROUND OF THE DISCLOSURE

Recently, “smart rings” have been developed and have become a popular consumer electronic device. From the outside, smart rings appear to be regular decorative rings. However, these smart rings may include wireless capabilities that allow them to pair with corresponding POS device for making payments. Also, some smart rings may instead be configured to pair with a smart phone. The wireless capabilities of these various smart rings require that antennas be incorporated into the ring. However, since some smart rings may be made of metal, it can be challenging to design and integrate antennas into the metallic rings. Typical designs on the market use chip antennas which require dedicated antenna volume that may already be scarce. Normally, these chip antennas have low performance as they typically rely on ground currents of very small Printed Circuit Board (PCB). Therefore, there is a need in the field of POS devices and smart rings to improve wireless communication with external devices.

Furthermore, some smart rings may include sensors for sensing biological parameters of the wearer. For example, smart rings may be configured to measure heart rate or pulse (e.g., in units of “beats for minute”). In this respect, a smart ring may include a heart rate sensing apparatus, which may include a Photoplethysmography (PPG) detecting device as well as processing capabilities for interpreting the PPG waveforms.

However, due to movement of a smart ring on the finger of a wearer and other situations, noise may be generated along with the true PPG signals. Thus, the detected PPG waveforms may include spikes, artifacts, and other types of noise that might be incorrectly interpreted as a heartbeat. Some conventional systems have attempted to correct for noise. However, these correction strategies often shift the phase of the detected signal, which can lead to problems in the frequency domain. Therefore, there is a need in the field of PPG sensing to provide improved correction strategies that not only can produce accurate data points, but also can maintain accurate timing features to prevent phase shifting of the data points.

### BRIEF SUMMARY OF THE DISCLOSURE

The present disclosure relates to systems and methods for performing photoplethysmography (PPG) analysis. Also, the present disclosure is related to PPG sensors, including smart rings with PPG sensors incorporated therein and processing for PPG detection. A process, according to one implementation, is configured for correcting data points of time intervals (e.g., IBI values) in a PPG dataset. The process includes a step of obtaining a sequence of data points (x) representing time interval calculations (y), each time interval calculation (y) being based on a length of time between two successive peaks (p) in a photoplethysmography (PPG) waveform, and each peak (p) being presumed to represent a volumetric blood-flow peak during one of multiple cycles of the PPG waveform. In response to detecting one or more spurious data points in a segment of the sequence, the process further includes a step of creating one or more artificial data points for replacing the one or more spurious data points, wherein the one or more artificial data points are derived using the time interval calculation (y) of each of the one or more spurious data points. More particularly, the replacing step may be performed in a manner to prevent an undesired phase shift in the frequency domain.

The process may further include analyzing each data point to determine if it is spurious and then correcting this data point (along with others in a specific segment of the sequence). The process may include determining whether any single spurious data point xₙ exceeds a previous data point xₙ₋₁ by more than a predetermined threshold. If so, the process includes deriving multiple artificial data points from the single spurious data point xₙ.

For example, deriving the multiple artificial data points may include a) calculating an average (avg) of the time interval calculations (y) between the previous data point xₙ₋₁ and a following data point xₙ₊₁, b) dividing the single spurious data point xₙ by the calculated average (avg) and rounding to the nearest integer (I), and c) replacing the single spurious data point xₙ by (I) artificial data points. The process may then include utilizing a smoothing technique to smoothly transition the (I) artificial data points from the previous data point xₙ₋₁ to the following data point xₙ₊₁.

The process may also include determining if one or more consecutive spurious data points xₙ, xₙ₊₁, . . . , x_{n+N-1} in the segment of the sequence fall below a previous data point xₙ₋₁ by more than a predetermined threshold. If so, the process may include deriving one or more artificial data points from (N) consecutive spurious data points xₙ, xₙ₊₁, . . . , x_{n+N-1} by.

For example, deriving these one or more artificial data points in this scenario may include a) calculating an average (avg) of the time interval calculations (y) between a prior data point and a later data point, b) adding the time interval calculations (y) of at least the (N) consecutive spurious data points xₙ, xₙ₊₁, . . . , x_{n+N-1} to obtain an accumulated sum, c) dividing the accumulated sum by the average (avg) and rounding to the nearest integer (I), and d) replacing original data points including at least the (N) consecutive spurious data points xₙ, xₙ₊₁, . . . , x_{n+N-1} by (I) artificial data points. The adding step b) may include adding the time interval calculations (y) of the (N) consecutive spurious data points xₙ, xₙ₊₁, . . . , x_{n+N-1} and one or more of a previous data point xₙ₋₁ and a following data point x_{n+N} to obtain the accumulated sum, where the replacing step d) may include replacing the (N) consecutive spurious data points xₙ, xₙ₊₁, . . . , x_{n+N-1} and the one or more of the previous data point xₙ₋₁ and the following data point x_{n+N} by the (I) artificial data points. The process may also include utilizing the smoothing technique to smoothly transition the (I) artificial data points from the prior data point xₙ-_to the later data point xₙ₊₁.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a graph illustrating an electrocardiography (ECG) waveform representing electrical activity of a healthy human heart measured near the heart itself, the graph also showing a first type of measurable peak-to-peak interval used for calculating heart rate, according to various embodiments.

FIG. 2 is a graph illustrating a Photoplethysmography (PPG) waveform representing volumetric blood-flow measurements of a healthy human heart measured at the extremities of the human body, the graph also showing a second type of measurable peak-to-peak interval used for calculating heart rate, according to various embodiments.

FIG. 3 is a bar graph illustrating Inter-Beat Interval (IBI) calculations related to a sequence of the second type of measurable peak-to-peak intervals used for calculating heart rate, according to various embodiments.

FIGS. 4 and 5 are bar graphs illustrating a number of conventional strategies for correcting spurious IBI calculations.

FIG. 6 is a diagram illustrating a data point correction technique for correcting a data point that exceeds a predetermined upper threshold, according to various embodiments of the present disclosure.

FIG. 7 is a diagram illustrating another data point correction technique for correcting a single data point that falls below a predetermined lower threshold, according to various embodiments of the present disclosure.

FIGS. 8A-8D are diagrams illustrating data point correction techniques for correcting multiple consecutive data points that fall below a predetermined lower threshold, according to various embodiments of the present disclosure.

FIG. 9 is a flow diagram illustrating a method for correcting data points related to PPG sensing, according to various embodiments.

FIG. 10 is a diagram of a smart ring.

FIG. 11A is a diagram illustrating an isometric, partially cut-away view of the smart ring of FIG. 2, according to various embodiments.

FIG. 11B is a diagram illustrating a cross-section side view of the smart ring of FIG. 2, according to various embodiments.

FIG. 12 is a diagram illustrating a system where a smart ring can wirelessly communicate with a POS machine and a mobile device, according to various embodiments of the present disclosure.

FIG. 13 is a block diagram of a user device, e.g., a mobile device.

FIGS. 14A-14C are screenshots of the GUI from the application illustrating management of the smart ring.

### DETAILED DESCRIPTION OF THE DISCLOSURE

The present disclosure relates to systems and methods for sensing biological characteristics of a person. According to various embodiments, heartbeat (or pulse) is detected, which can then be used to determine various types of medical conditions for the person. In particular, the embodiments of the present disclosure are configured to obtain raw data from one or more sensors attached to the person's body or used in other suitable ways for sensing heart and blood flow activities.

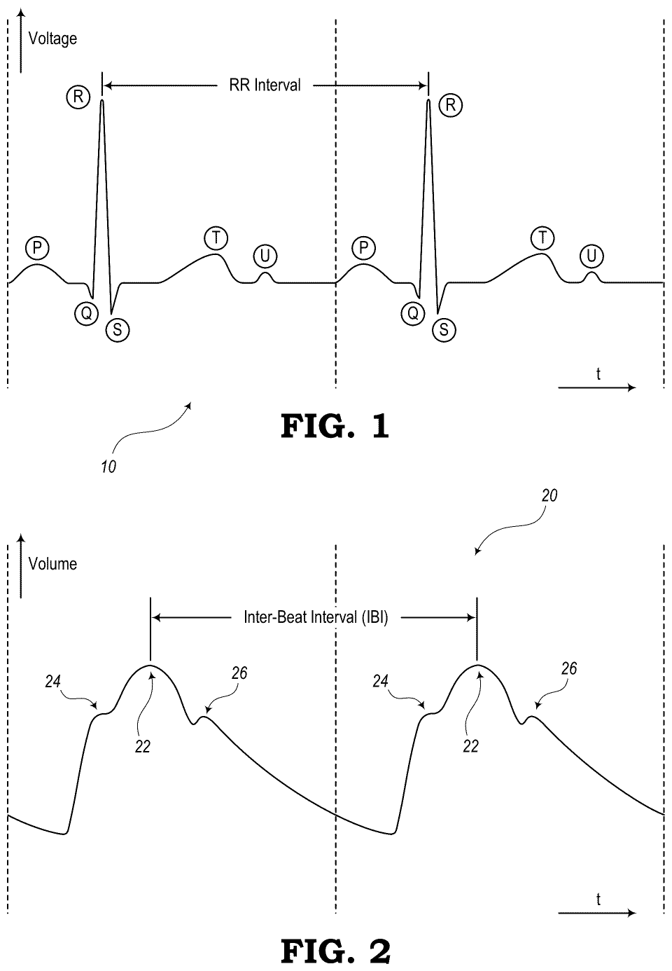

FIG. 1 is a graph illustrating an example of an electrocardiography (ECG) waveform 10 representing electrical activity of a healthy human heart. In particular, the ECG waveform 10 shows the detection of voltage over time. The ECG waveform 10 may be measured by attaching one or more ECG leads to the chest and arms of the person. Thus, the ECG waveform 10 may represent the voltage detected at any one of the leads. For simplicity, FIG. 1 shows only two periods of the ECG waveform 10.

In a normal heart, electrical activity includes depolarization (or contraction) and repolarization (or relaxation) of the heart muscles. As shown, the ECG waveform 10 indicates the detection of several different electrical events, which are based on the contraction and relaxation of specific sets of heart muscles related to the atrial and ventricular chambers of the heart as well as the activities of other sets of muscles for opening and closing heart valve, whereby the activities of all of these sets are muscles are coordinated to pump blood throughout the body.

In particular, the ECG waveform 10 includes a P wave, a QRS complex, a T wave, and a U wave. The letters P, Q, R, S, T, U are commonly known as arbitrary variables that, more importantly, are used for designating different portions of the ECG waveform 10. The P wave is an upward deflection that represents atrial depolarization. The QRS complex includes a downward dip (Q), followed by an upward spike (R), followed by another downward dip (S). The QRS complex represents ventricular depolarization. The R wave is significant with respect to measuring heartbeat. For example, a peak-to-peak interval (referred to as the “RR interval”) can be measured from the peak of the R wave in one cardiac cycle to the peak of the next R wave in the next cardiac cycle. The RR interval is the time elapsed between two successive R waves of the QRS signal on the ECG. The T wave is an upward deflection that represents ventricular repolarization. The U wave is a slight upward deflection (sometimes imperceptible) that represents papillary muscle repolarization.

By measuring several cardiac cycles, it is possible to detect many different characteristics of the human heart, including possible defects. Also, a measure of heartbeat (or pulse) can be detected by simply taking the inverse of the time interval (e.g., RR interval) between consecutive peaks, usually measured in milliseconds. For example, to measure the heartbeat in beats per minute (bpm), the time interval (in msec) is inverted and then multiplied by 60000. A more accurate heartbeat can be measured by averaging several consecutive interval calculations. The heartrate, which is a reciprocal of the RR interval calculations, is a function of intrinsic properties of the sinus node of a patient as well as autonomic influences.

It may be noted, however, that achieving an ECG waveform, such as the one shown in FIG. 1, requires that the electrical leads are placed on the bare chest and arms of a patient, which thereby restricts the patient's movement. Therefore, less restrictive methods have been developed for measuring heartbeat. For example, Photoplethysmography (PPG) analysis provides a simpler method for calculating heartbeat.

### Photoplethysmography (PPG) Sensing

FIG. 2 is a graph illustrating an example of a PPG waveform 20 for a healthy human heart. The PPG waveform 20 represents volumetric blood-flow measurements measured through the veins, such as at the extremities of the human body. Thus, instead of an electrical (e.g., voltage) measurement as is done with ECG, the PPG waveform 20 is a measure of the changes in the volume of blood flowing through a blood vessel. It should be noted that the PPG waveform 20 may be measured at different parts of the body and may therefore produce waveforms having different shapes and characteristics. According to the example of the PPG waveform 20 shown in FIG. 2, each cycle may include a true peak 22 plus other characteristics, such as a first false peak 24 and a second false peak 26, where the false peaks 24 and 26 might incorrectly be interpreted as the true peak 22.

The PPG waveform 20 may be measured by shining an LED light (e.g., green LED) onto the skin of the person where a blood vessel is detectable below the skin. Then, the PPG device uses a photosensor for sensing the light reflection. In some respects, the true peaks 22 of the PPG waveform 20 may correspond (with some time delay) to the peak of the R wave of the ECG waveform 10. That is, with the occurrence of the major pumping action of the heart, represented by the R wave, blood is forced throughout the body and can then be detected in the form of volume changes in the blood vessel, as shown in the PPG waveform 20.

To measure heartrate, the PPG waveform 20 may also be analyzed to detect the peak-to-peak intervals from one cycle to the next. In the PPG environment, the peak-to-peak interval is referred to as an “Inter-Beat Interval” and represents the time interval between beats (or pulses) between consecutive waveform cycles. It should be noted that this second type of measurable peak-to-peak interval (i.e., IBI interval) for calculating heartrate will normally be about the same as the first peak-to-peak interval (i.e., RR interval) that might be obtained using the ECG measurement technique. Again, by considering a sequence of IBI intervals, it is possible to accurately detect an average heartrate for the person, as well as other heart characteristics, such as Heart Rate Variability (HRV).

The following descriptions of heartrate monitoring are generally associated with measurements using various types of PPG sensors. According to some embodiments, PPG sensors may be incorporated into smart watches and other portable devices for enabling easy use by a person and avoiding the complications and limitations of ECG measurements. In some embodiments, PPG sensors may also be incorporated into smart rings. As such, a person can wear a smart ring on his or her finger, essentially at all times, thereby allowing his or her heartrate to be monitored continuously.

When the PPG sensor is being developed or customized for a particular individual or user, a specific threshold value may be established for detecting a peak within each pulse cycle. When the PPG signal exceeds the threshold value and then reaches a peak (e.g., when the first derivative or slope of the PPG signal is zero), this event may be interpreted as a peak (e.g., true peak 22) within that cycle. This peak, along with other peaks, is used for the calculation of the IBI value.

However, it should be noted that errors may occur in the detection of the PPG signals. For example, if a person moves his or her finger (or hand) and/or moves the smart ring with respect to the finger, certain motion artifacts (e.g., noise, spikes, etc.) may arise. These artifacts may reach a level that rises above the threshold levels and therefore may be interpreted as a peak. Also, if false peaks (e.g., false peaks 24, 26, etc.) rise above the threshold levels, these too may incorrectly be interpreted as the true peak 22. This may happen, for example, if the detected PPG signal is stronger than normal. Thus, at times, the processing of data from a PPG sensor may incorrectly record these extra peaks (e.g., external or environmental spikes, noise events, artifacts, high false peaks, etc.) as heartbeats, which of course may cause errors in later calculations. In this case, the step of calculating IBI values when extra peaks are picked up results in multiple time intervals (e.g., IBI calculations) that are well below other normal IBI calculations. Therefore, in order to account for these excessive peaks resulting in very low IBI calculation, the embodiments of the present disclosure are configured to handle this situation.

In addition, there may be occurrences when the detected PPG signal is weaker than normal. In this case, an actual blood-flow volume peak (e.g., true peak 22) may not reach the established threshold level and may therefore be missed. Other events (e.g., positioning the PPG sensor too far from the skin, external light on the PPG sensor, etc.) can cause weak PPG signals resulting in missed peaks. In this case, the step of calculating IBI values when fewer peaks are picked up results in an extremely long time interval (e.g., IBI calculation) that is well above other normal IBI calculations. Therefore, in order to account for one or more missed peaks resulting in a very high IBI calculation, the embodiments of the present disclosure are configured to handle this situation as well.

Therefore, the present disclosure is configured to provide various embodiments for working around these errors, artifacts, disturbances, misinterpretations, etc. In particular, the embodiments of the present disclosure may include the steps of detecting peaks in the PPG signals, calculating time intervals between successive peaks in a waveform, and recording these calculated intervals. Then, according to the present embodiments, instead of simply accepting the calculated intervals (e.g., IBI intervals), the present disclosure processes to the intervals to determine if there are any apparent inconsistencies that can be attributed to sensing errors. When these discrepancies are detected, the present disclosure corrects the errors and then outputs clean data. Also, the cleaned data also represents data that does not fluctuate with time. That is, the data points are not expanded or compressed, which is common in conventional systems. Instead, the embodiments of the present disclosure are configured to reduce (or eliminate) phase shift errors caused by expanding or compacting data into longer or shorted times.

### Dataset of Peak-to-Peak Interval Calculations

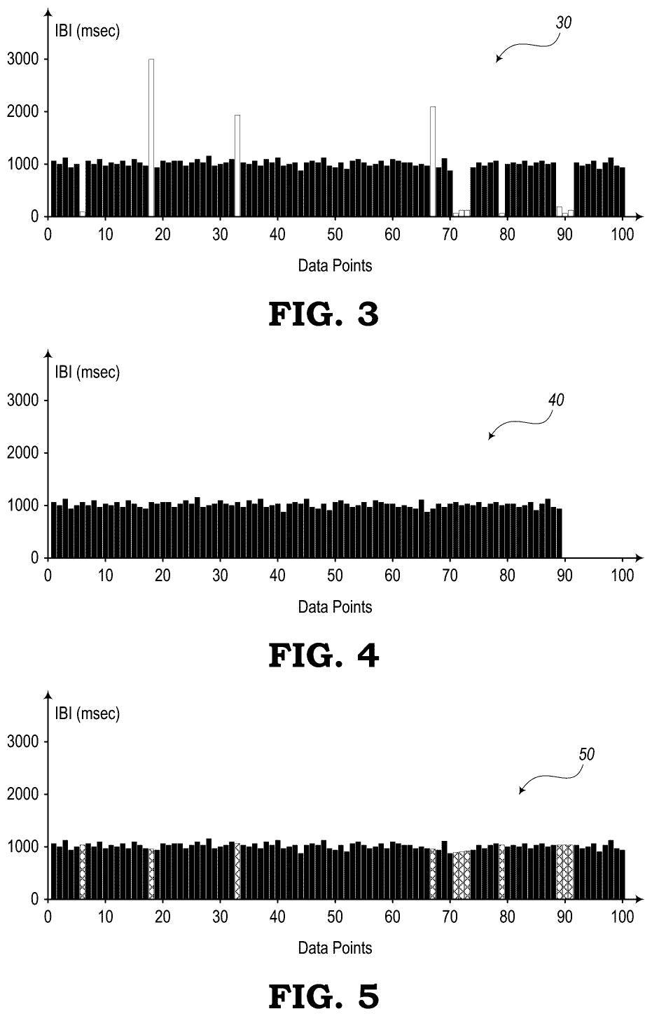

FIG. 3 is a bar graph 30 illustrating an example of data points calculated over time for a person. Each data point represents an Inter-Beat Interval (IBI) calculation (in msec) based on the time between consecutive peaks in a PPG signal (i.e., peak-to-peak calculation) using a PPG sensor. As shown in FIG. 3, the bar graph 30 includes 100 samples of data points obtained over a certain period of time (e.g., about 100 seconds). The data points can be used for calculating heartrate and/or other heart-related parameters (e.g., HRV).

From the bar graph 30, it may be understood that the peak-to-peak intervals (IBIs), throughout the entire test duration, remains near 1000 msec. That is, each beat (or pulse) is detected about every second, which may represent a healthy resting heartrate of about 60 bpm. Data points on the bar graph 30 that appear to be normal (or without error) are shown in black, while data points that appear to be abnormal are shown in white. Thus, the abnormal data points (white bars) represent spurious (or ectopic) values and are determined to include values that are outside of a normal range. For example, the data points at sample positions 6, 71, 72, 73, 79, 89, 90, and 91 include values far below the other samples. Also, the data points at sample positions 18, 33, and 67 include values far above the other samples.

FIGS. 4 and 5 are bar graphs illustrating a number of conventional strategies for correcting spurious IBI calculations. For example, FIG. 4 shows a bar graph 40 that results from using a method that involves completing eliminating or deleting the spurious values. Thus, the under-values (at positions 6, 71, 72, 73, 79, 89, 90, and 91) as well as the over-values (at positions 18, 33, and 67) in the bar graph 30 of FIG. 3 are completely removed. However, a problem with this method is that the 100 total samples of FIG. 3 are reduced down to 89 samples in this example. Hence, there is a significant amount of time (during the entire test) that is discarded, resulting in a phase shift in the frequency domain that can result in calculation errors when the dataset of the bar graph 40 is to be further processed for analysis of heartbeat and/or other heart-related measurements.

FIG. 5 shows a bar graph 50 where a combination of various conventional “interpolation” methods may be performed to correct for the detection of spurious IBI calculations. These conventional methods may include the process of simply replacing each spurious data point with a value derived from nearby good samples. For example, some conventional strategies may include Nearest Neighbor Interpolation (NNI), Linear Interpolation (LI), Cubic Spline Interpolation (CSI), and various combinations of these. In this way, each of the conventional methods ignores (and removes) what had been detected and calculated (i.e., the actual IBI values) and replaces the gaps with values that are unrelated to the actual data points.

For example, with respect to the samples at positions 89, 90, and 91, it should be understood that the time intervals from the bar graph 30 of FIG. 3 shows that very little time had actually passed from the neighboring good samples (from sample 88 to sample 92). However, according to conventional methods shown in the bar graph 50 of FIG. 5, this small amount of time has been replaced with three interpolated samples of about 1000 msec each. That is, it may be noted that an extra 3000 msec (3 seconds) has been added in the bar graph 50. Also, the time interval (IBI) at position 18 appears to be close to 3000 msec, but it is replaced with an interpolated value of about 1000 msec, thereby removing about two seconds from the data sequence. Therefore, the conventional interpolation methods may be inclined to interrupt the real world timeline of data samples by inserting extra time where it does not belong or removing time where it should exist. This discrepancy can cause various inconsistencies and further processing errors, such as issues with phase shifting in the frequency domain.

Therefore, instead of deleting spurious data points (as is done in the conventional method of FIG. 4) or replacing spurious data points with interpolated values (as is done in the conventional methods of FIG. 5), the embodiments of the present disclosure use specific strategies for achieving usable data points. Instead of ignoring the spurious data points, the techniques of the present disclosure are configured to use the actual values of spurious data points. That is, although a time interval may not accurately depict a heartrate or blood-flow event, it still should be recognized as a real time measurement. For example, if a peak is missed (or if multiple consecutive peaks are missed), the next detected time interval value will essentially be about twice as high as (or multiple times) what it should be if the peak were not missed. Thus, instead of deleting (or interpolating) the approximately 3000 msec data point at position 18, the present disclosure determines the value of the excessive value and determines that it should more accurately be interpreted as three 1000 msec intervals.

Also, with respect to one or multiple very low data points (e.g., at sample positions 89, 90, 91), the present disclosure may be configured to add up the time intervals to determine that certain noise events, motion artifacts, etc. may have been introduced in the PPG waveform that were incorrectly interpreted as peaks. Therefore, instead of assuming that each spurious data point needs to be replaced with an interpolated value close to the adjacent values, the present disclosure adds up the accumulate time of the time intervals and may determine that the one or more small intervals may more accurately be interpreted as a single interval with noise and artifacts mixed in and can therefore replace the multiple samples with fewer samples.

### Replacing Spurious Inter-Beat Interval (IBI) Data Points

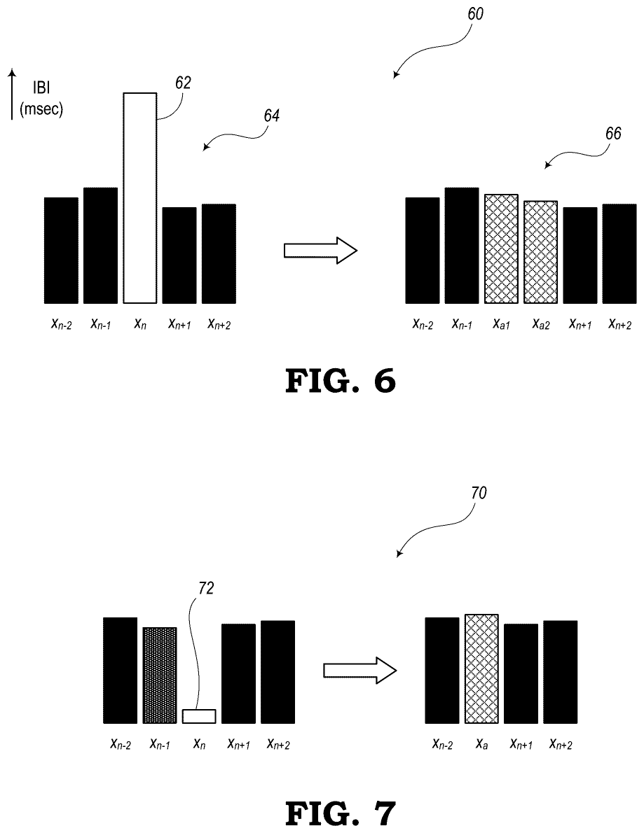

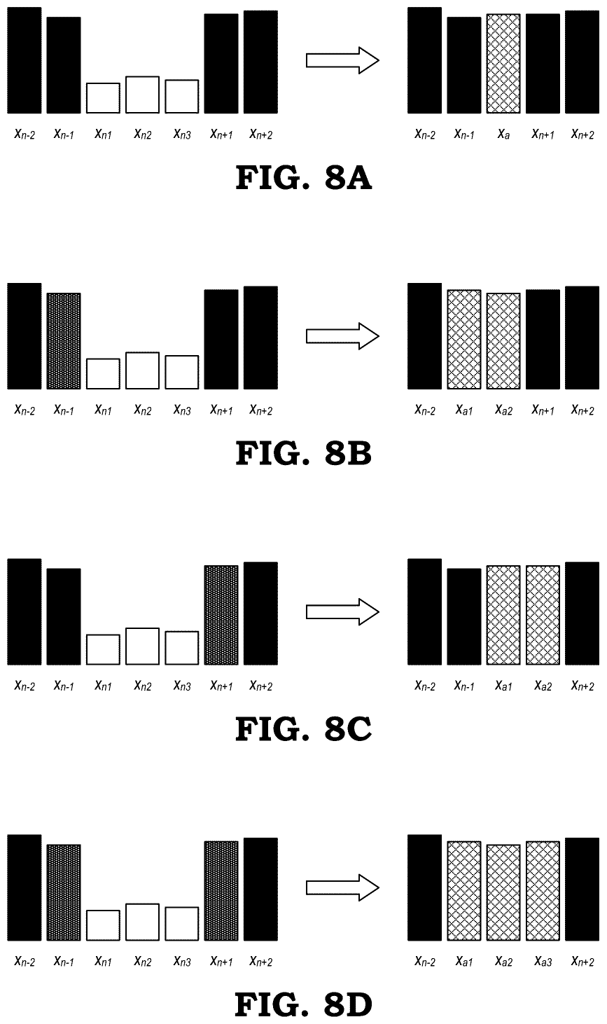

FIG. 6 is a diagram illustrating an example of a data point correction technique 60 for correcting a single data point 62 that exceeds a predetermined upper threshold. For example, this predetermined upper threshold may be equal to a certain percentage (e.g., 20%) above the previous data point. The original data points 64 (e.g., IBI values), such as those shown in the bar graph 30 of FIG. 3, are shown on the left side of FIG. 6. After the specific processing, as described in the present disclosure, the data points are converted to replacement values 66 as shown on the right side of FIG. 6. The same arrangement (i.e., the original data points depicted on the left and the replacement data points depicted on the right) is repeated throughout FIGS. 7 and 8 as well.

In the examples illustrated in FIGS. 6-8, the variable “x” is used to represent data points and the subscript “n” is used to represent index numbers of the data points. Thus, the sequence of data points shown in FIG. 6, for example, includes the first spurious data point 62 represented by “xₙ,” where the two data points before this one are represented as xₙ₋₂ and xₙ₋₁, and where the two data points after this one are represented as xₙ₊₁ and xₙ₊₂ in this segment of the data point sequence. On the right side, after processing, “artificial” data points are represented by indices “a.” In FIG. 6, two artificial data points are inserted and are represented by the variables xₐ₁ and xₐ₂. In this case, the other data points xₙ₋₂, xₙ₋₁, xₙ₊₁, and xₙ₊₂ remain the same.

Therefore, a condition is detected where the value (in msec) of a data point xₙ exceeds the upper threshold limit, the next data point (xₙ₊₁) is analyzed to make sure it is properly within an acceptable range (s 20% of the last acceptable data point xₙ₋₁). The embodiments of the present disclosure may then perform the following steps to replace the spurious data point xₙ.
a) Calculate the average time interval between the previous good data point (xₙ₋₁) and the next good data point (xₙ₊₁), where

avg=(xₙ₋₁+xₙ₊₁)/2 and
b) Divide the value of the spurious data point (xₙ) by the average (xₙ/avg) and round to the nearest integer (I).

In the example of FIG. 6, the value (e.g., about 2000 msec) of the spurious data point 62 (xₙ) is calculated as being about twice as high as the average values (e.g., about 1000 msec) of the two adjacent points. Thereby the integer value (I) is calculated as 2 in this example. Thus, two artificial data points are inserted in place of the single spurious data point 62. When added together, the two artificial data points xₐ₁ and xₐ₂ may be equal to the same value as the original value of the spurious data point 62. In some embodiments, a smoothing procedure may be used to smoothly transition from the first adjacent data point xₙ₋₁ to the second adjacent data point xₙ₊₁.

It may be noted that the five data points on the left are replaced by the six data points on the right. That is, an extra data point is added. The index numbers “n,” which were arbitrarily assigned for the purpose of illustration in this and the following examples, may be changed for reference purposes as needed. Also, it should further be noted that the 100 samples shown in FIG. 3 may actually be an inaccurate number of actual peaks and may therefore be change to more data points (as in the embodiment of FIG. 6) or fewer data points (as in the embodiments of FIGS. 7 and 8).

FIG. 7 is a diagram illustrating an example of another data point correction technique 70 for correcting a single data point 72 that falls well below a predetermined lower threshold (e.g., 20% below the previous data point). In this case in which only a single extremely small IBI value is present in the sequence of data points, it may be assumed that a single noise spike was detected as a peak and thereby divided one actual time interval into two smaller time intervals.

To correct this issues according to one embodiment, the time interval value of the faulty data point xₙ may be added to the value of the previous data point xₙ₋₁ to obtain an accumulated time. An artificial data point xₐ is then added in place of the data points xₙ and xₙ₋₁, whereby the artificial data point xₐ has a value equal to the accumulated time. Thus, the five data points shown on the left are converted to the four data points shown on the right and the actual time measurement is preserved. That is, the actual values of the replaced data points are not ignored, but instead are used for the calculation of the artificial data point xₐ.

In other embodiments with respect to FIG. 7, instead of replacing the previous data point xₙ₋₁, the following data point xₙ₊₁ may be replaced (along with the faulty data point xₙ), whereby the values of these two data points may be added to form the artificial data point. In still other embodiments, three data points xₙ₋₁, xₙ, and xₙ₊₁ may be replaced by two artificial data points, whereby the values of these three data points may be added and then divided by two to form the two artificial data points.

FIGS. 8A-8D are diagrams illustrating embodiments of data point correction techniques for correcting multiple consecutive data points that fall below a predetermined lower threshold. For example, in these figures, the three consecutive very-low data points xn1, xn2, and xn3 represents IBI time intervals (in msecs) may be calculated, where it may be determined that the reason for the spurious values is that they were likely cause by excessive false peaks or spikes in the PPG readings.

The values of each of the faulty data points may be added together. In some cases, this total may further be added with the values of one or both of the previous and following data points and then averaged accordingly. This total time sum (in msec), which again represent real time information, can then be used to reconstruct artificial data points that better represent real heartbeat events and not simply a correction of a dataset with faulty data points. Also, the artificial data points described herein use the actual time information to correct the data points while also avoiding the removal of actual time and/or introduction of extra time into the real timeline represented by the dataset. Thus, the embodiments of the present disclosure are configured to avoid the issues of the conventional systems that tend to shift the phase in time causing problems in the frequency domain and resulting in problems with future processing of the dataset.

In FIG. 8A, a first correction technique is demonstrated. The accumulated sum total of the time values (in msec) of the three spurious data points xₙ₁, xₙ₂, xₙ₃ (e.g., about 300 msec, 360 msec, and 340 msec, respectively) may be equal to about 1000 msec. Then, an average is calculated between the previous “good” data point xₙ₋₁ and the following “good” data point xₙ₊₁, where

avg=(xₙ₋₁+xₙ₊₁)/2

In this example, assume that avg is equal to about 1000 msec. Then, the accumulated sum is divided by avg and rounded to the nearest integer (I), which in this example would be 1. Thus, an artificial data point xₐ is inserted in the sequence between xₙ₋₁ and xₙ₊₁ and is given the value of the accumulated sum (e.g., about 1000 msec). Again, no time is lost in the reconstructing of the artificial data point.

In FIG. 8B, a second correction technique is demonstrated with respect to cases where three spurious data points xₙ₁, xₙ₂, xₙ₃ are detected. This strategy may be used, for example, in cases where it may be known that a slightly lower than normal IBI value is followed by a much lower than normal IBI value, such as when the first false peak 24 of the PPG signal 20 of FIG. 2 may tend to be detectable as a true peak.

In this example, an accumulated sum total of the time values (in msec) of the previous “good” data point xₙ₋₁ and three spurious data points xₙ₁, xₙ₂, xₙ₃ is calculated. Assume, for example, that these values are about 1000 msec, about 300 msec, 360 msec, and 340 msec, respectively. Thus, the total will be equal to about 2000 msec. Then, an average is calculated between a previous boundary data point xₙ₋₂ and the following “good” data point xₙ₊₁, where

avg=(xₙ₋₂+xₙ₊₁)/2

In this example, assume that avg is equal to about 1000 msec. Then, the accumulated sum is divided by avg and rounded to the nearest integer (I), which in this example would be two (2). Thus, two artificial data points xₐ₁ and xₐ₂ are inserted in the sequence between the boundary points xₙ₋₂ and xₙ₊₁ and is given the value of the accumulated sum (e.g., about 2000 msec) divided by I (2). In this case, the two artificial data points may each be equal to about 1000 msec. Again, no time is lost in the reconstructing of the artificial data point. Also, as described with respect to FIG. 6, a smoothing procedure may be used to smoothly transition from the first adjacent or boundary data point xₙ₋₂ to the second adjacent or boundary data point xₙ₊₁.

In FIG. 8C, a third correction technique is demonstrated with respect to cases where three spurious data points xₙ₁, xₙ₂, xₙ₃ are detected. This strategy is similar to the strategy described with respect to FIG. 8B except that instead of grouping the previous data point xₙ₋₁ with the three spurious data points, the following data point xₙ₊₁ is grouped with the spurious data points. The strategy of FIG. 8C may be used, for example, in cases where it may be known that a much lower than normal IBI value is followed by a slightly lower than normal IBI value, such as when the second false peak 26 of the PPG signal 20 of FIG. 2 may tend to be detectable as a true peak.

In this example, an accumulated sum total of the time values (in msec) of the three spurious data points xₙ₁, xₙ₂, xₙ₃ and the following “good” data point xₙ₊₁ is calculated. Assume, for example, that these values are about 300 msec, 360 msec, 340 msec, and about 1000 msec, respectively. Thus, the total will be equal to about 2000 msec. Then, an average is calculated between a previous “good” data point xₙ₋₁ and the following boundary data point xₙ+₂, where

avg=(xₙ₋₁+xₙ₊₂)/2

In this example, assume that avg is equal to about 1000 msec. Then, the accumulated sum is divided by avg and rounded to the nearest integer (I), which in this example would be two (2). Thus, two artificial data points xₐ₁ and xₐ₂ are inserted in the sequence between the boundary points xₙ₋₁ and xₙ₊₂ and is given the value of the accumulated sum (e.g., about 2000 msec) divided by I (2). In this case, the two artificial data points may each be equal to about 1000 msec. Again, no time is lost in the reconstructing of the artificial data point. Also, as described with respect to FIG. 6, a smoothing procedure may be used to smoothly transition from the first adjacent or boundary data point xₙ₋₁ to the second adjacent or boundary data point xₙ₊₂.

In FIG. 8D, a fourth correction technique is demonstrated with respect to cases where three spurious data points xₙ₁, xₙ₂, xₙ₃ are detected. In this example, an accumulated sum total of the time values (in msec) of the previous “good” data point xₙ₋₁, the three spurious data points xₙ₁, xₙ₂, xₙ₃, and the following “good” data point xₙ₊₁ is calculated. Assume, for example, that these values are about 1000 msec, about 300 msec, 360 msec, 340 msec, and about 1000 msec, respectively. Thus, the total will be equal to about 3000 msec. Then, an average is calculated between a previous boundary data point xₙ₋₂ and the following boundary data point xₙ₊₂, where

avg=(xₙ₋₂+xₙ₊₂)/2

In this example, assume that avg is equal to about 1000 msec. Then, the accumulated sum is divided by avg and rounded to the nearest integer (I), which in this example would be three (3). Thus, three artificial data points xₐ₁, xₐ₂, and xₐ₃ are inserted in the sequence between the boundary points xₙ₋₂ and xₙ₊₂ and is given the value of the accumulated sum (e.g., about 3000 msec) divided by I (3). In this case, the three artificial data points may each be equal to about 1000 msec. Again, no time is lost in the reconstructing of the artificial data point. Also, as described with respect to FIG. 6, a smoothing procedure may be used to smoothly transition from the first adjacent or boundary data point xₙ₋₂ to the second adjacent or boundary data point xₙ₊₂.

Various types of smoothing procedures or algorithms may be used to artificially smooth the artificially reconstructed data points. In one embodiment, a smoothing procedure may include the following:
a) Calculate a step size of inserting IBI values between to the previous (left) adjacent boundary data point and the following (right) adjacent boundary data point.
b) Compare the value of the left and right adjacent boundary data points to determine whether the inserted IBI values should be incremented or decremented.
c) Calculate the inserted sample positions accordingly.
d) Replace the original samples in the segment with the new IBI values.
e) Concatenate the IBI values outside the segment and the IBI values inside the segment to compose a new peak sequence.

Photoplethysmography (PPG) is an uncomplicated and inexpensive optical measurement method that may be used for heart rate monitoring purposes. PPG is a non-invasive technology that uses a light source and a photodetector at the surface of the skin of a patient to measure the volumetric variations of blood circulation.

Except for cases in which a person has an abnormal heartbeat, such as a premature beat, each IBI value may normally be calculated as varying from a previous IBI value by less than about 5%. Therefore, the 20% threshold values for both the upper limit and the lower limit may be set to values closer to 5% in order to detect more spurious data points and perform the correction strategies mentioned with respect to FIGS. 6-8.

Regarding various advantages of the embodiments of the present disclosure, Heart Rate Variation (HRV) can be calculated in different ways. The frequency domain calculation method, such as LF/HF, is very sensitive to the high frequency domain noise that is introduced from inaccurate heart beat detection. The traditional way to detect the abnormal heart beat is to compare the IBI value with the average value of a duration. These conventional methods are not accurate if the heart rate varies greatly during this period. The methods of the present disclosure may be similar to linear prediction techniques based on one or more previous heart beats.

Many of the conventional systems remove the spurious data from the time sequence for the calculation of HRV. Removing may not have a significant impact on statistical calculations, such as SDNN, or RMSSD, but will cause a phase shift for the rest of the following data. This may introduce extra frequency domain noise when frequency domain parameters are calculated. On the other hand, the embodiments of the present disclosure remove the spurious data, but then insert artificial results back into the time sequence. Thus, the artificial replacement strategies discussed herein prevent the phase shift for the rest of the data results and reduce the noise in the frequency domain when HRV and other heart conditions are calculated.

The embodiments of the present disclosure may utilize any suitable type of PPG sensors for measuring at least the peaks of the PPG waveform. Based on the times of the peaks, the IBI can be calculated. Then, a sequence of data points (e.g., as shown in FIG. 3) may be generated. This dataset can then be analyzed according to the present strategies and replacements can be made for any spurious data points.

Some types of PPG sensors (along with the various processing) may be dedicated devices simply for measuring heartbeat or heartrate. In other embodiments, PPG sensors may be incorporated into other devices, such as smart watches, smart rings, etc., which may have other functions as well.

### Data Point Correction Method

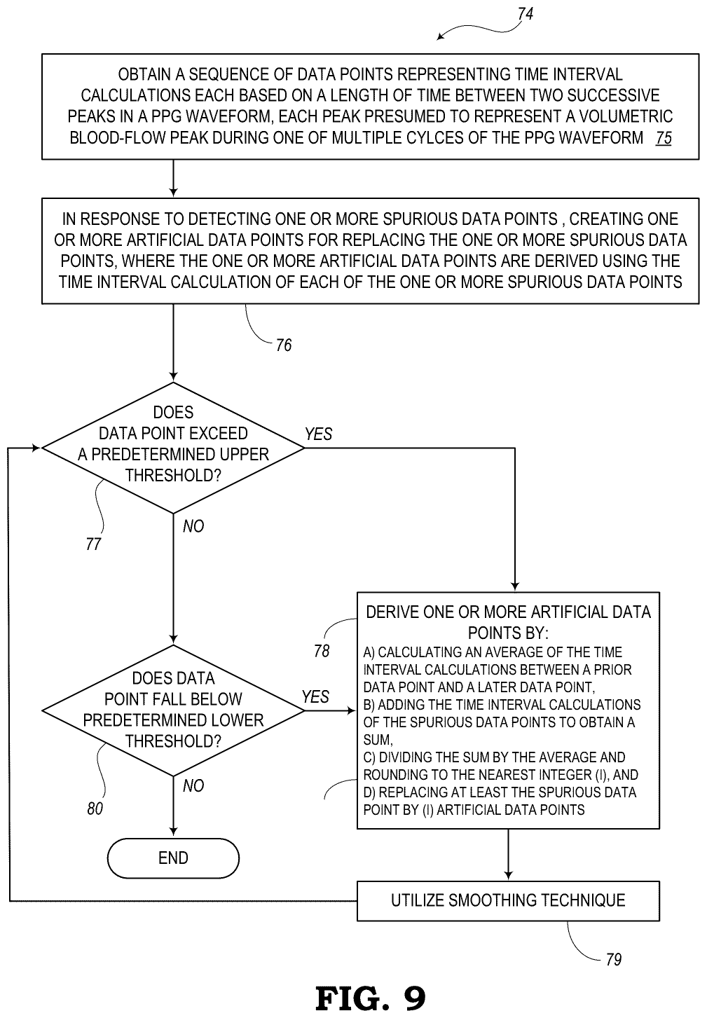

FIG. 9 is a flow diagram illustrating an embodiment of a process 74 for correcting data points of time intervals (e.g., IBI values) in a dataset. The process 74 includes a step obtaining a sequence of data points (x) representing time interval calculations (y), as indicated in block 75, wherein each time interval calculation (y) is based on a length of time between two successive peaks (p) in a photoplethysmography (PPG) waveform, and each peak (p) is presumed to represent a volumetric blood-flow peak during one of multiple cycles of the PPG waveform. In response to detecting one or more spurious data points in a segment of the sequence, the process 74 further includes a step of creating one or more artificial data points for replacing the one or more spurious data points, as indicated in block 76, wherein the one or more artificial data points are derived using the time interval calculation (y) of each of the one or more spurious data points. As a result, the replacing step (block 76) may be performed in a manner to prevent an undesired phase shift in the frequency domain.

The process 74 may further include analyzing each data point to determine if it is spurious and then correcting this data point (along with others in a specific segment of the sequence). The process 74 may include determining whether any single spurious data point xₙ exceeds a previous data point xₙ₋₁ by more than a predetermined threshold, as indicated in condition block 77. If so, the process 74 goes to block 78, which includes deriving multiple artificial data points from the single spurious data point xₙ.

Deriving the multiple artificial data points may include a) calculating an average (avg) of the time interval calculations (y) between the previous data point xₙ₋₁ and a following data point xₙ₊₁, b) dividing the single spurious data point xₙ by the calculated average (avg) and rounding to the nearest integer (I), and c) replacing the single spurious data point xₙ by (I) artificial data points. The process 74 may then proceed to block 79, which may include utilizing a smoothing technique to smoothly transition the (I) artificial data points from the previous data point xₙ₋₁ to the following data point xₙ₊₁.

If it is determined in condition block 77 that the single spurious data point xₙ does not exceed the previous data point xₙ₋₁ by more than the first predetermined threshold, the process 74 may proceed to condition block 80, which includes determining if one or more consecutive spurious data points xₙ, xₙ₊₁, . . . , x_{n+N-1} in the segment of the sequence fall below a previous data point xₙ₋₁ by more than a predetermined threshold. If not, the process ends for the tested data point. However, if one or more data points fall below, the process 74 proceeds to block 78, which includes deriving one or more artificial data points from (N) consecutive spurious data points xₙ, xₙ₊₁, . . . , x_{n+N-1} by.

Deriving these one or more artificial data points may include a) calculating an average (avg) of the time interval calculations (y) between a prior data point and a later data point, b) adding the time interval calculations (y) of at least the (N) consecutive spurious data points xₙ, xₙ₊₁, . . . , x_{n+N-1} to obtain an accumulated sum, c) dividing the accumulated sum by the average (avg) and rounding to the nearest integer (I), and d) replacing original data points including at least the (N) consecutive spurious data points xₙ, xₙ₊₁, . . . , x_{n+N-1} by (I) artificial data points. The adding step b) may include adding the time interval calculations (y) of the (N) consecutive spurious data points xₙ, xₙ₊₁, . . . , x_{n+N-1} and one or more of a previous data point xₙ₋₁ and a following data point x_{n+N} to obtain the accumulated sum, where the replacing step d) may include replacing the (N) consecutive spurious data points xₙ, xₙ₊₁, . . . , x_{n+N-1} and the one or more of the previous data point xₙ₋₁ and the following data point x_{n+N} by the (I) artificial data points. The process 74 may then proceed to block 79, which may include utilizing a smoothing technique to smoothly transition the (I) artificial data points from the prior data point xₙ₋₁ to the later data point xₙ₊₁.

In the process 74, the predetermined threshold is divided into upper one and lower one, and block 78 is responsible for calculating the number (I) of artificial data points accordingly. In the process 74, the predetermined threshold is the absolute value of PPI variation rate such as 20% or 15%. If there are consecutive big PPIs that PPI variation rates are bigger than the (upper) threshold, the summation is also needed to obtain the number (I). In addition, the process 74 would check block 77/80 again after smoothing and do block 78 if necessary.

### Smart Ring

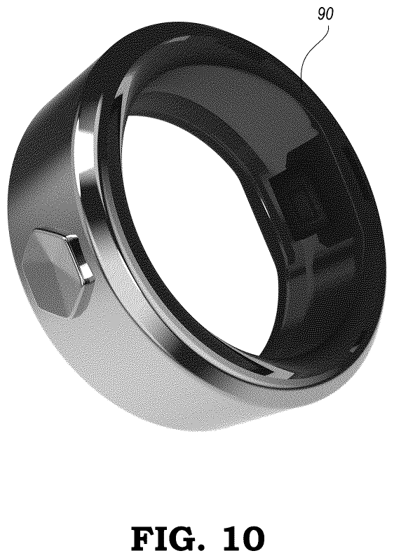

FIG. 10 is a diagram illustrating an embodiment of a smart ring 90. The smart ring 90 can include a titanium finishing, a light weight, and a slim profile (e.g., <3.5 mm thick, 8 mm width), can be water resistant, have a one-week battery life, have various sizes, etc. In an embodiment, the smart ring 90 can include various sensors, such as a 14-bit Photoplethysmography (PPG) sensing device and a three-axis accelerometer. The smart ring 90 can be configured to measure vitals such as heart rate, heart rate variability, sleep, activity, falls, and the like.

The smart ring 90 may include a rechargeable battery for powering components in the device, including the PPG sensor. A charging case having a lid and looks/works a bit like a jewelry box may be used for charging the smart ring 90. In an embodiment, the charging case can have no buttons, lights, or anything on the charger. The charging case can include a hexagonal shape.

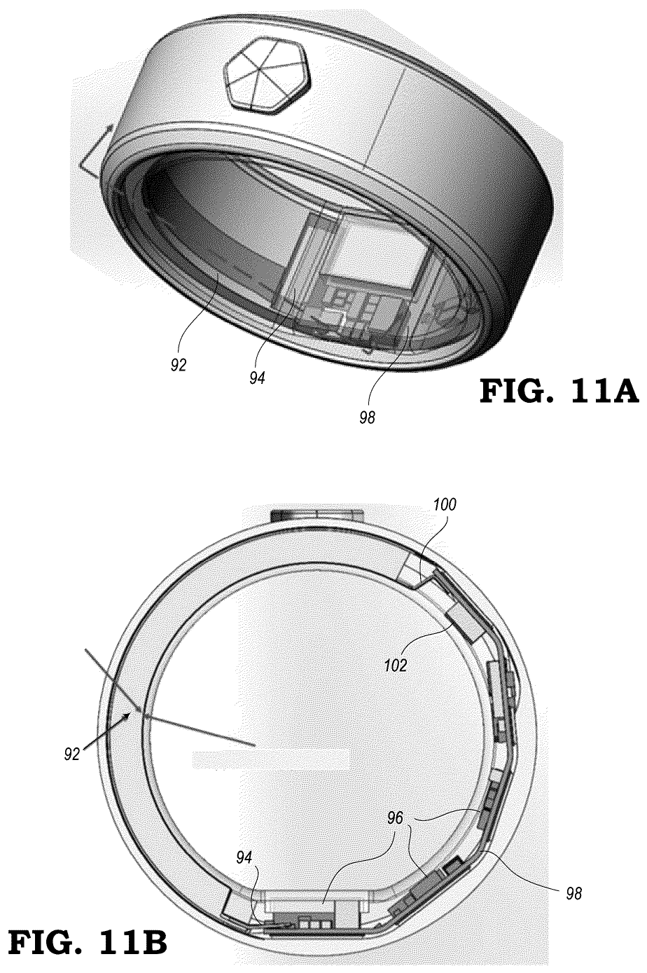

FIG. 11A is a diagram illustrating an isometric, partially cut-away view of an embodiment of the smart ring 90 of FIG. 10. Also, FIG. 11B is a diagram illustrating a cross-section side view of the smart ring 90. A battery casing 92 may be embedded in the smart ring 90 and may be connected via a first set of connections 94 to electrical circuits 96 formed on a Flexible Printed Circuit (FPC) 98. A second set of connections 100 is formed between the other end of the battery casing 92 and the other end of the FPC 98 and is used for connection to another electrical circuit 102, which may also be formed on the FPC 98.

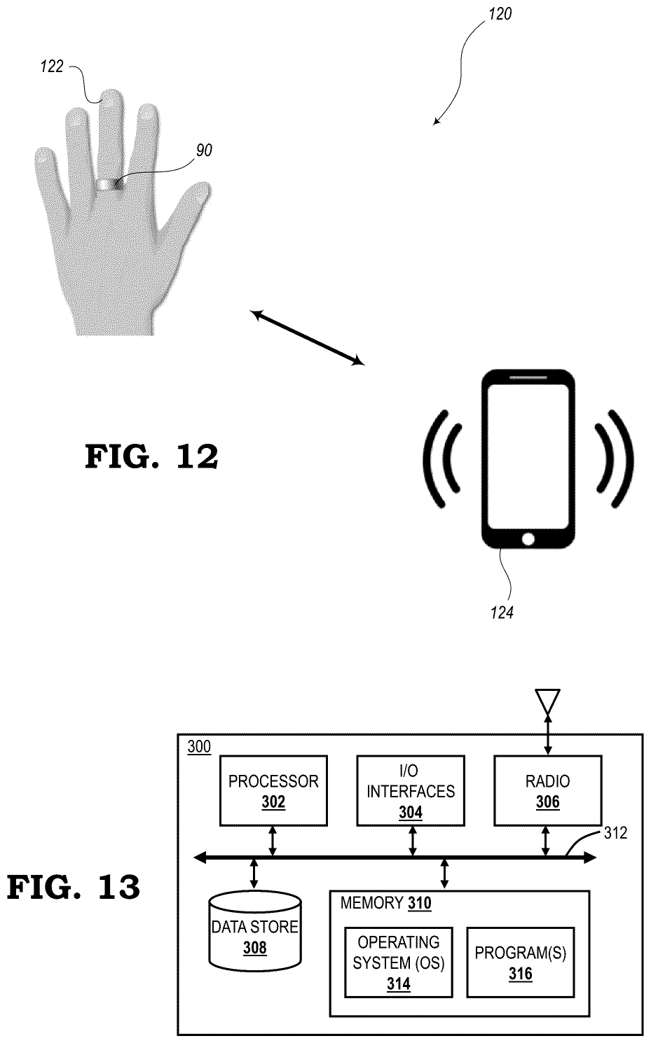

FIG. 12 is a diagram illustrating a system 120 in which the smart ring 90 can wirelessly communicate at short range to various devices. For example, the smart ring 90 may be worn on a finger 122 of a user or patient. When positioned near a mobile device 124, for example, the smart ring 90 and mobile device 124 may be configured to operate within a first frequency band (e.g., Bluetooth frequencies) to enable communication therebetween. When positioned close to other devices (e.g., a Point-of-Sale (POS) machine), the smart ring 90 and POS machine may be configured to operate within a second frequency band (e.g., Near Field Communication (NFC) frequencies) to enable communication therebetween.

Conventional smart rings normally do not allow operation within two separate frequency bands. However, according to the various embodiments of the present disclosure, various antenna components of the smart ring 90 include specific physical characteristics and electrical circuitry that enable operation at two different frequency band. This allows the smart ring 90 to pair with the mobile device 124 to enable operation within the first frequency band (e.g., Bluetooth) while also allowing the smart ring 90 to pair with the POS machine or other NFC equipment to enable operation within the second frequency band (e.g., NFC). In particular, antennas (and antenna segments or portions) may be configured to be fully embedded in a normal-sized ring, such as the smart ring 90. These antenna portions may include, for example, the electrically conductive battery casing 92 and also a conductive trace or film on the FPC 98 or other suitable flexible board that can be embedded within the normal-sized ring. By using these components, which may already be needed for wireless communication, it may be possible to minimize the extra number of parts and circuitry to conserve space within the outer shell of the smart ring 90.

### User Device Architecture

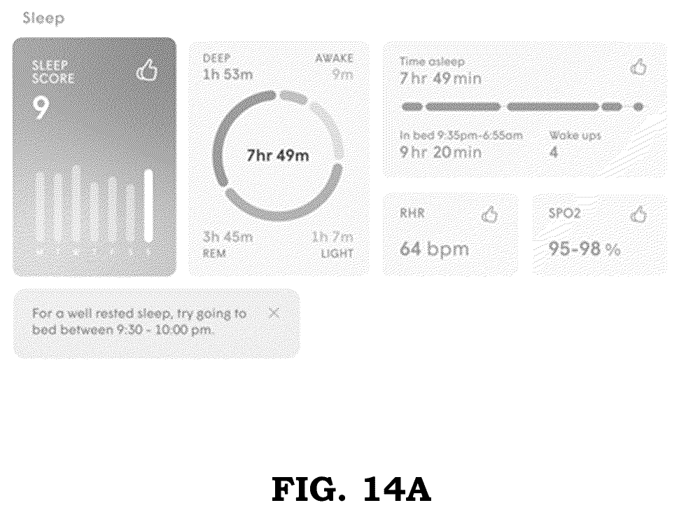

FIG. 13 is a block diagram of a user device 300 (e.g., PPG sensor, smart ring 90, etc.). The user device 300 can be a digital device that, in terms of hardware architecture, generally includes a processor 302, input/output (I/O) interfaces 304, a radio 306, a data store 308, and memory 310. It should be appreciated by those of ordinary skill in the art that FIG. 13 depicts the user device 300 in an oversimplified manner, and a practical embodiment may include additional components and suitably configured processing logic to support known or conventional operating features that are not described in detail herein. Also, the user device 300 may be referred to as a mobile device, smart phone, tablet, smart watch, etc. The components (302, 304, 306, 308, and 302) are communicatively coupled via a local interface 312. The local interface 312 can be, for example, but not limited to, one or more buses or other wired or wireless connections, as is known in the art. The local interface 312 can have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 can be any custom made or commercially available processor, a central processing unit (CPU), an auxiliary processor among several processors associated with the user device 300, a semiconductor-based microprocessor (in the form of a microchip or chipset), or generally any device for executing software instructions. When the user device 300 is in operation, the processor 302 is configured to execute software stored within the memory 310, to communicate data to and from the memory 310, and to generally control operations of the user device 300 pursuant to the software instructions. In an embodiment, the processor 302 may include a mobile optimized processor such as optimized for power consumption and mobile applications. The I/O interfaces 304 can be used to receive user input from and/or for providing system output. User input can be provided via, for example, a keypad, a touch screen, a scroll ball, a scroll bar, buttons, a barcode scanner, and the like. System output can be provided via a display device such as a liquid crystal display (LCD), touch screen, and the like. The I/O interfaces 304 can also include, for example, a serial port, a parallel port, a small computer system interface (SCSI), an infrared (IR) interface, a radio frequency (RF) interface, a universal serial bus (USB) interface, and the like. The I/O interfaces 304 can include a graphical user interface (GUI) that enables a user to interact with the user device 300. Additionally, the I/O interfaces 304 may further include an imaging device, i.e., camera, video camera, etc.

The radio 306 enables wireless communication to an external access device or network. Any number of suitable wireless data communication protocols, techniques, or methodologies can be supported by the radio 306. The data store 308 may be used to store data. The data store 308 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media.

The memory 310 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 310 may incorporate electronic, magnetic, optical, and/or other types of storage media. Note that the memory 310 may have a distributed architecture, where various components are situated remotely from one another but can be accessed by the processor 302. The software in memory 310 can include one or more software programs, each of which includes an ordered listing of executable instructions for implementing logical functions. In the example of FIG. 3, the software in the memory 310 includes a suitable operating system (O/S) 314 and programs 316. The operating system 314 essentially controls the execution of other computer programs and provides scheduling, input-output control, file and data management, memory management, and communication control and related services. The programs 316 may include various applications, add-ons, etc. configured to provide end-user functionality with the user device 300. For example, example programs 316 may include, but not limited to, a web browser, social networking applications, streaming media applications, games, mapping and location applications, electronic mail applications, financial applications, and the like. In a typical example, the end user typically uses one or more of the programs 316 along with a network.

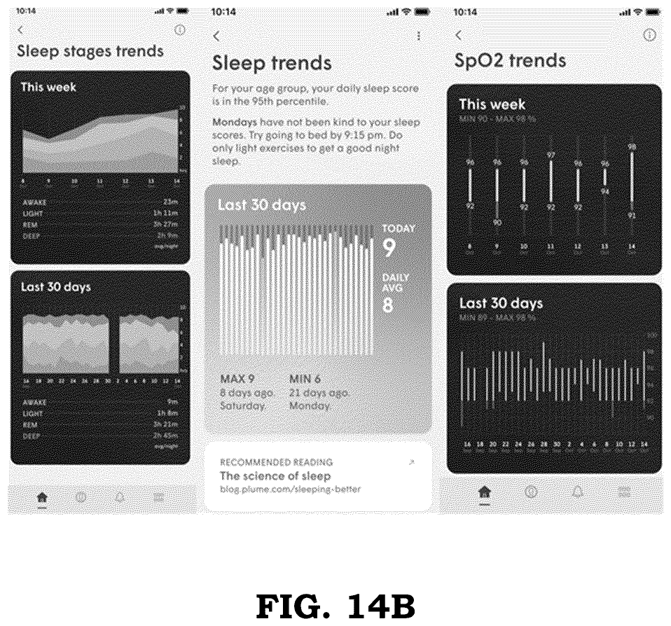

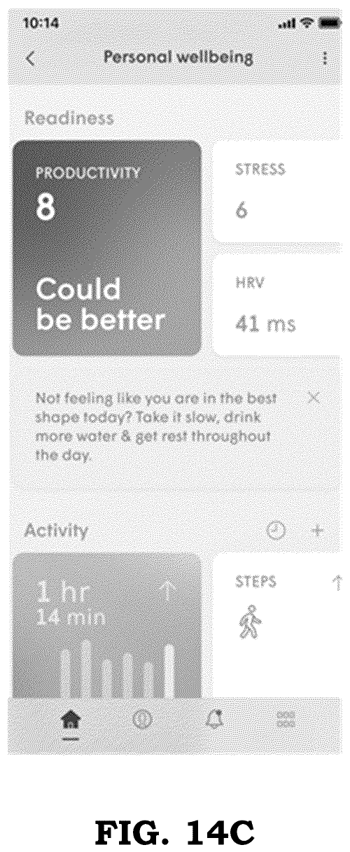

FIGS. 14A-14C are screenshots of a Graphical User Interface (GUI), display screen, or other suitable user output element that may be associated with various types of PPG sensors or the smart ring 90. The screenshots of the GUI from the application illustrate management of the smart ring 90 or PPG sensor. Advantageously, the application can combine management off the distributed Wi-Fi system 200 and the smart ring 90 in a single GUI.

In FIGS. 14A-14C, the application can include an activity view, trend notifications, graphs, etc. The application, for the smart ring 90, can be a personal health coach that monitors your health and gives you personalized recommendations to stay healthy, in conjunction with the smart ring 90, the distributed Wi-Fi system 200, and the user device 300. This can provide AI-driven health monitoring. The application can support Fall detection, Sleep trend and stress detection via Heart Rate Variability (HRV), Blood oxygen trends, Activity and over activity monitoring, Heart rate monitoring, Care group notification, Blood pressure monitoring, body temperature, stress, and the like.

### Use Cases

The smart ring 90 may include a battery, memory, processing circuitry, a plurality of sensors, a plurality of antennas, and a battery, each coupled to one another and all enclosed in a casing (e.g., titanium), wherein the processing circuitry is configured to cause the smart ring to pair with any of a user device and a Wi-Fi access point in a distributed Wi-Fi system, and obtain measurements from any of the plurality of sensors and provide the measurements to a cloud service, via the any of the user device and the Wi-Fi access point. The components of the smart ring 90 may work together for connectivity to external devices, namely the cloud 212 service, the distributed Wi-Fi system 200, the user device 300, third-party devices, Point-of-Sale (PoS) devices, and the like. The following are non-limiting use cases with the smart ring 90 and other components. The smart ring 90 may measure SpO2 (oxygen saturation) via a PPG sensor that looks at wavelengths that are absorbed, reflected from blood flow in the finger. The smart ring 90 may also set and monitor values with respect to thresholds. The various sensors (e.g., PPG sensors) in the smart ring 90 can be used to alert when various thresholds are detected, such as SpO2 min, heartrate max, etc. The notification can include push notifications, third-party notifications (caregiver, guardian, etc.), via the application on the user device 300, an audible alarm for thresholds, a visual indicator, a haptic/vibration indicator, etc.

### CONCLUSION

It will be appreciated that some embodiments described herein may include or utilize one or more generic or specialized processors (“one or more processors”) such as microprocessors; Central Processing Units (CPUs); Digital Signal Processors (DSPs): customized processors such as Network Processors (NPs) or Network Processing Units (NPUs), Graphics Processing Units (GPUs), or the like; Field-Programmable Gate Arrays (FPGAs); and the like along with unique stored program instructions (including both software and firmware) for control thereof to implement, in conjunction with certain non-processor circuits, some, most, or all of the functions of the methods and/or systems described herein. Alternatively, some or all functions may be implemented by a state machine that has no stored program instructions, or in one or more Application-Specific Integrated Circuits (ASICs), in which each function or some combinations of certain of the functions are implemented as custom logic or circuitry. Of course, a combination of the aforementioned approaches may be used. For some of the embodiments described herein, a corresponding device in hardware and optionally with software, firmware, and a combination thereof can be referred to as “circuitry configured to,” “logic configured to,” etc. perform a set of operations, steps, methods, processes, algorithms, functions, techniques, etc. on digital and/or analog signals as described herein for the various embodiments.

Moreover, some embodiments may include a non-transitory computer-readable medium having instructions stored thereon for programming a computer, server, appliance, device, at least one processor, circuit/circuitry, etc. to perform functions as described and claimed herein. Examples of such non-transitory computer-readable medium include, but are not limited to, a hard disk, an optical storage device, a magnetic storage device, a Read-Only Memory (ROM), a Programmable ROM (PROM), an Erasable PROM (EPROM), an Electrically EPROM (EEPROM), Flash memory, and the like. When stored in the non-transitory computer-readable medium, software can include instructions executable by one or more processors (e.g., any type of programmable circuitry or logic) that, in response to such execution, cause the one or more processors to perform a set of operations, steps, methods, processes, algorithms, functions, techniques, etc. as described herein for the various embodiments.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims. Moreover, it is noted that the various elements, operations, steps, methods, processes, algorithms, functions, techniques, etc. described herein can be used in any and all combinations with each other.

What is claimed is:

1. A method comprising the steps of:
obtaining a sequence of data points (x) representing time interval calculations (y), each time interval calculation (y) based on a length of time between two successive peaks (p) in a photoplethysmography (PPG) waveform, each peak (p) presumed to represent a volumetric blood-flow peak during one of multiple cycles of the PPG waveform; and
in response to detecting one or more spurious data points in a segment of the sequence, creating one or more artificial data points for replacing the one or more spurious data points, wherein the one or more artificial data points are derived using the time interval calculation (y) of each of the one or more spurious data points.

2. The method of claim 1, wherein replacing the one or more spurious data points with the one or more artificial data points is performed in a manner to prevent an undesired phase shift in the frequency domain.

3. The method of claim 1, further comprising the step of determining that a single spurious data point xₙ in the segment of the sequence is outside of a normal range if the single spurious data point xₙ exceeds a previous data point xₙ₋₁ by more than a predetermined threshold.

4. The method of claim 3, further comprising the step of deriving multiple artificial data points from the single spurious data point xₙ by:
calculating an average (avg) of the time interval calculations (y) between the previous data point xₙ y and a following data point xₙ₊₁,
dividing the single spurious data point xₙ by the calculated average (avg) and rounding to the nearest integer (I), and
replacing the single spurious data point xₙ by (I) artificial data points.

5. The method of claim 4, further comprising the step of utilizing a smoothing technique to smoothly transition the (I) artificial data points from the previous data point xₙ₋₁ to the following data point xₙ₊₁.

6. The method of claim 1, further comprising the step of determining that a number (N) of consecutive spurious data points xₙ, xₙ₊₁, . . . , x_{n+N-1} in the segment of the sequence is outside of a normal range if each spurious data point falls below a previous data point xₙ₋₁ by more than a predetermined threshold.

7. The method of claim 6, wherein one or more artificial data points are derived from the (N) consecutive spurious data points xₙ, xₙ₊₁, . . . , x_{n+N-1} by:
calculating an average (avg) of the time interval calculations (y) between a prior data point and a later data point,
adding the time interval calculations (y) of at least the (N) consecutive spurious data points xₙ, xₙ₊₁, . . . , x_{n+N-1} to obtain an accumulated sum,
dividing the accumulated sum by the average (avg) and rounding to the nearest integer (I), and
replacing original data points including at least the (N) consecutive spurious data points xₙ, xₙ₊₁, . . . , x_{n+N-1} by (I) artificial data points.

8. The method of claim 7, wherein the adding step includes adding the time interval calculations (y) of the (N) consecutive spurious data points xₙ, xₙ₊₁, . . . , x_{n+N-1} and one or more of a previous data point xₙ₋₁ and a following data point x_{n+N} to obtain the accumulated sum, and wherein the replacing step includes replacing the (N) consecutive spurious data points xₙ, xₙ₊₁, . . . , x_{n+N-1} and the one or more of the previous data point xₙ₋₁ and the following data point x_{n+N} by the (I) artificial data points.

9. The method of claim 7, further comprising the step of utilizing a smoothing technique to smoothly transition the (I) artificial data points from the prior data point to the later data point.

10. The method of claim 1, further comprising the steps of:
detecting the PPG waveform using an LED source and a photodetector;
analyzing the PPG waveform to determine a plurality of events presumed to represent multiple volumetric blood flow peaks during the multiple cycles of the PPG waveform;
recording a point in time when each event is determined; and
determining the time interval calculations based on the difference between the points in time between each successive pair of events.

11. The method of claim 1, wherein the time interval calculations are Inter-Beat Intervals (IBIs) and are inversely related to heart rate.

12. The method of claim 1, wherein the one or more spurious data points are the result of misinterpreting peaks in the PPG waveform, missing actual volumetric blood-flow peaks, signal noise, and movement artifacts.

13. A smart ring comprising:
a photoplethysmography (PPG) sensor for obtaining a PPG waveform from a wearer of the smart ring; and
a processing device configured to
obtain a sequence of data points (x) representing time interval calculations (y), each time interval calculation (y) based on a length of time between two successive peaks (p) in the PPG waveform, each peak (p) presumed to represent a volumetric blood-flow peak of the wearer during one of multiple cycles of the PPG waveform, and
in response to detecting one or more spurious data points in a segment of the sequence, create one or more artificial data points for replacing the one or more spurious data points, wherein the one or more artificial data points are derived using the time interval calculation (y) of each of the one or more spurious data points.

14. The smart ring of claim 13, wherein the processing device is further configured to determine that a single spurious data point xₙ in the segment of the sequence is outside of a normal range if the single spurious data point xₙ exceeds a previous data point xₙ₋₁ by more than a predetermined threshold.

15. The smart ring of claim 14, wherein the processing device is further configured to derive multiple artificial data points from the single spurious data point xₙ by:
calculating an average (avg) of the time interval calculations (y) between the previous data point xₙ₋₁ and a following data point xₙ₊₁,
dividing the single spurious data point xₙ by the calculated average (avg) and rounding to the nearest integer (I), and
replacing the single spurious data point xₙ by (I) artificial data points.

16. The smart ring of claim 13, wherein the processing device is further configured to determine that a number (N) of consecutive spurious data points xₙ, xₙ₊₁, . . . , x_{n+N-1} in the segment of the sequence is outside of a normal range if each spurious data point falls below a previous data point xₙ₋₁ by more than a predetermined threshold.

17. The smart ring of claim 16, wherein the processing device is configured to derive one or more artificial data points from the (N) consecutive spurious data points xₙ, xₙ₊₁, . . . , x_{n+N-1} by:
calculating an average (avg) of the time interval calculations (y) between a prior data point and a later data point,
adding the time interval calculations (y) of at least the (N) consecutive spurious data points xₙ, xₙ₊₁, . . . , x_{n+N-1} to obtain an accumulated sum,
dividing the accumulated sum by the average (avg) and rounding to the nearest integer (I), and
replacing original data points including at least the (N) consecutive spurious data points xₙ, xₙ₊₁, . . . , x_{n+N-1} by (I) artificial data points.

18. The smart ring of claim 17, wherein the processing device is configured to accumulate the sum by adding the time interval calculations (y) of the (N) consecutive spurious data points xₙ, xₙ₊₁, . . . , x_{n+N-1} and one or more of a previous data point xₙ₋₁ and a following data point x_{n+N}, and wherein the processing device is configured to replace the original data points by replacing the (N) consecutive spurious data points xₙ, xₙ₊₁, . . . , x_{n+N-1} and the one or more of the previous data point xₙ₋₁ and the following data point x_{n+N} by the (I) artificial data points.

19. The smart ring of claim 13, wherein the PPG sensor includes an LED source and a photodetector for detecting the PPG waveform, and wherein the processing device is further configured to:
analyze the PPG waveform to determine a plurality of events presumed to represent multiple volumetric blood-flow peaks during the multiple cycles of the PPG waveform;
record a point in time when each event is determined; and
determine the time interval calculations based on the difference between the points in time between each successive pair of events.

20. The smart ring of claim 13, wherein the time interval calculations are Inter-Beat Intervals (IBIs) and are inversely related to a heart rate of the wearer, and wherein the one or more spurious data points are the result of misinterpreting peaks in the PPG waveform, missing actual volumetric blood-flow peaks, signal noise, and motion artifacts.
